(19) United States
(12) Patent Application Publication
(10) Pub. No.: US 2019/0333646 A1
(43) Pub. Date: Oct. 31, 2019

(54) SYSTEMS AND METHODS FOR DETERMINING, TRACKING, AND PREDICTING COMMON INFECTIOUS ILLNESS OUTBREAKS

(71) Applicant: KNOX SPENCER ASSOCIATES LLC, Moon Township, PA (US)

(72) Inventor: Daniel F. Shaw, Moon Township, PA (US)

(21) Appl. No.: 16/309,378

(22) PCT Filed: Jul. 14, 2017
(86) PCT No.: PCT/US17/42121

§ 371 Date: Dec. 12, 2018

Related U.S. Application Data
(60) Provisional application No. 62/362,608, filed on Jul. 15, 2016.

(51) Int. Cl. G16H 50/80

(52) U.S. Cl. CPC G16H 50/80

(57) ABSTRACT

Methods and systems for tracking common infectious illnesses and disseminating information are disclosed. A computer-based method of tracking a common infectious illness and disseminating information regarding the common infectious illness to a plurality of users via one or more of a mobile device and a user computing device includes receiving data from one or more electronic sources; determining the common infectious illness from the data; determining one or more of a location and a frequency of the common infectious illness from the data; and plotting information relating to the common infectious illness on a map. The information includes a current severity of the common infectious illness in a particular area and predicted trend of the severity of the common infectious illness The method further includes providing, by the processing device, the map to the plurality of users.

### CROSS-REFERENCE TO RELATED APPLICATION

The present application claims priority to U.S. Provisional Patent Application Ser. No. 62/362,608, filed Jul. 15, 2016 and entitled “Systems and Methods for Determining, Tracking, and Predicting Common Illness Outbreaks,” the entire contents of which is incorporated herein by reference.

### TECHNICAL FIELD

The present specification generally relates systems and methods for monitoring common infectious illness diagnoses and, more specifically, to systems and methods for determining, tracking, and predicting a common infectious illness outbreak.

### BACKGROUND

Common infectious illnesses can be a nuisance in the sense that they disrupt home schedules and cause individuals to miss school and/or work. In addition, common infectious illnesses can exacerbate serious illnesses or other health problems. As such, individuals may desire to avoid contracting such common infectious illnesses and may go to certain lengths to avoid coming into contact with others that have the illness or are exhibiting symptoms thereof.

Because common infectious illnesses are typically non-life threatening and can be very prevalent at times, public health authorities generally do not focus their efforts on tracking such illnesses and such illnesses are generally not officially reported. Rather, public health authorities tend to focus on more debilitating diseases and illnesses that can result in mortality, birth defects, serious injury, and/or the like. Systems and methods that are related to tracking common infectious illness generally access self-reporting data, such as social network data and/or patient complaint data, which can be unreliable because an individual may report that he/she has a particular illness when in fact he/she does not have that illness or has a different illness. In addition, it may be unreliable to track a location of a person having a particular illness via self-reporting data.

Accordingly, a need exists for systems and methods that determine, track, and predict common infectious illnesses using reliable data, such as medical coding and/or insurance databases.

### SUMMARY

In an embodiment, a computer-based method of tracking a common infectious illness and disseminating information regarding the common infectious illness to a plurality of users via one or more of a mobile device and a user computing device includes receiving, by a processing device, data from one or more electronic sources; determining, by the processing device, the common infectious illness from the data; determining, by the processing device, one or more of a location and a frequency of the common infectious illness from the data; and plotting, by the processing device, information relating to the common infectious illness on a map. The information includes a current severity of the common infectious illness in a particular area and predicted trend of the severity of the common infectious illness. The method further includes providing, by the processing device, the map to the plurality of users.

In another embodiment, a system for tracking a common infectious illness and disseminating information regarding the common infectious illness to a plurality of users via one or more of a mobile device and a user computing device includes a processing device and a non-transitory, processor-readable storage medium. The non-transitory, processor readable storage medium includes one or more programming instructions thereon that, when executed, cause the processing device to receive data from one or more electronic sources; determine the common infectious illness from the data; determine one or more of a location and a frequency of the common infectious illness from the data; and plot information relating to the common infectious illness on a map. The information comprises a current severity of the common infectious illness in a particular area and predicted trend of the severity of the common infectious illness. The programming instructions further cause the processing device to provide the map to the plurality of users.

In yet another embodiment, a computer-based method of tracking a plurality of common infectious illnesses and disseminating information regarding each common infectious illness from the plurality of common infectious illnesses to a plurality of users via one or more of a mobile device and a user computing device includes receiving, by a processing device, data from one or more electronic sources; determining, by the processing device, each common infectious illness from the data; determining, by the processing device, one or more of a location and a frequency of each common infectious illness from the data; and plotting, by the processing device, information relating to each common infectious illness on a map. The information includes a current severity of each common infectious illness in a particular area and predicted trend of the severity of each common infectious illness. The computer-based method further includes providing, by the processing device, the map to the plurality of users.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts an illustrative computing network according to one or more embodiments shown and described herein;

FIG. 2A schematically depicts a block diagram of illustrative hardware of a computing network according to one or more embodiments shown and described herein;

FIG. 2B schematically depicts a block diagram of software modules contained within a memory of a computing device according to one or more embodiments shown and described herein;

FIG. 2C schematically depicts a block diagram of various data contained within a data storage component of a computing device according to one or more embodiments shown and described herein;

FIG. 3 depicts a flow diagram of an illustrative method of tracking and predicting common infectious illness outbreaks according to one or more embodiments shown and described herein;

FIG. 4 depicts a flow diagram of an illustrative method of determining a location and frequency of an illness from received data according to one or more embodiments shown and described herein;

FIG. 5 depicts a screen shot of an illustrative user interface containing a map according to one or more embodiments shown and described herein;

FIG. 6 depicts a screen shot of an illustrative user interface containing a description of a common infectious illness according to one or more embodiments shown and described herein;

FIG. 7 depicts a screen shot of an illustrative user interface containing a chart of historical trends according to one or more embodiments shown and described herein;

FIG. 8 depicts a screen shot of an illustrative user interface containing forecast trends according to one or more embodiments shown and described herein; and

FIG. 9 depicts a screen shot of an illustrative user interface containing age group trends according to one or more embodiments shown and described herein.

### DETAILED DESCRIPTION

The embodiments described herein are generally directed to systems and methods that obtain data from medical, insurance, and/or public health related sources, determine common infectious illness information from the data, plot the common infectious illness information on a map, and predict an outbreak of the common infectious illness based on the plots on the map. The data may be collected over a period of time such that movement in the plots can be observed (e.g., certain areas are seeing an increase in a particular illness over a period of time). The systems and methods described herein can also be used to present the mapped information to one or more users (e.g., via a website, a mobile app and/or the like) so as to notify the one or more users of a predicted outbreak. In addition, the systems and methods described herein can provide the one or more users in an area where an outbreak currently exists or is predicted to exist with information on preventing contraction of the common infectious illness, treatment options, medical staff contact information, and/or the like.

As used herein, the term “common infectious illness” generally refers to illnesses that are frequently contracted by members of a population in a developed country. While such illnesses can be life threatening at least to a certain subset of the population, in general such illnesses are viewed more as a nuisance than a life threatening disease. That is, in general, an average member of the population can and does recover from the illness after being treated and/or after a certain period of time has elapsed. Such common infectious illnesses are generally contagious and can spread between individuals of a population. Illustrative examples of such common infectious illnesses include, but are not limited to, the common cold, bronchitis, bronchiolitis, gastroenteritis, mononucleosis, an ear infection, Lyme disease, otitis media (i.e., middle ear infection), acute sinusitis (i.e., sinus infection), streptococcal pharyngitis (i.e., strep throat), tonsillitis, upper respiratory infections such as laryngotracheobronchitis (i.e., croup), influenza (including type A flu and type B flu), pneumonia, or the like, conjunctivitis, methicillin-resistant Staphylococcus aureus (MRSA) infections, respiratory syncytial virus (RSV), and the like.

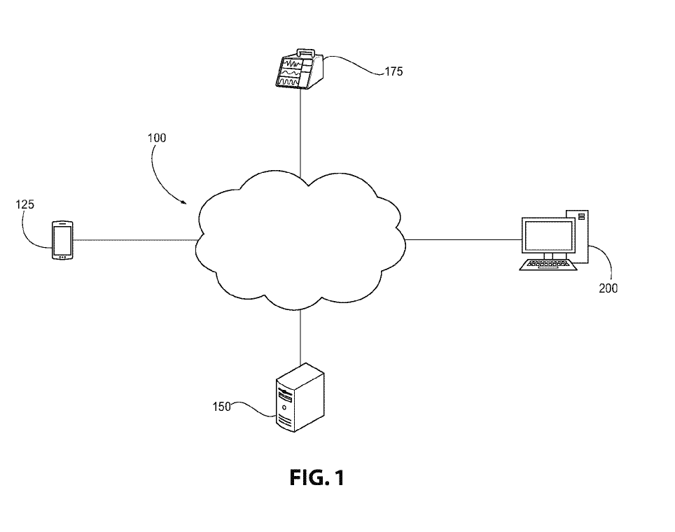

FIG. 1 depicts an illustrative computing network that depicts components for a system that obtains, tracks, and predicts common infectious illness outbreaks according to embodiments shown and described herein. As illustrated in FIG. 1, a computer network 100 may include a wide area network (WAN), such as the Internet, a local area network (LAN), a mobile communications network, a public service telephone network (PSTN), a personal area network (PAN), a metropolitan area network (MAN), a virtual private network (VPN), and/or another network. The computer network 100 may generally be configured to electronically connect one or more computing devices and/or components thereof. Illustrative computing devices may include, but are not limited to, a user computing device 200, a mobile computing device 125, and a server computing device 150.

The mobile computing device 125 and the user computing device 200 may each generally be used as an interface between a user and the other components connected to the computer network 100, and/or various other components communicatively coupled to the mobile computing device 125 and/or the user computing device 200 (such as components communicatively coupled via one or more networks to the mobile computing device 125 and/or the user computing device 200), whether or not specifically described herein. Thus, the mobile computing device 125 and/or the user computing device 200 may be used to perform one or more user-facing functions, such as receiving one or more inputs from a user or providing information to the user. Additionally, in the event that the server computing device 150 requires oversight, updating, or correction, the mobile computing device 125 and/or the user computing device 200 may be configured to provide the desired oversight, updating, and/or correction. The mobile computing device 125 and/or the user computing device 200 may also be used to input additional data into a data storage portion of the server computing device 150. Illustrative examples of the mobile computing device 125 and/or the user computing device 200 include a smartphone, a tablet, a personal computer, an Internet-connected user device (such as a smart watch, a fitness band, a personal assistant device, and the like), an Internet-connected consumer electronic device, and the like. In some embodiments, the mobile computing device 125 and/or the user computing device 200 may be a generic device that can be loaded with a software program, module, and/or the like to provide the functionality described herein. In other embodiments, the mobile computing device 125 and/or the user computing device 200 may be a specialized device that is particularly designed and configured to provide the functionality described herein.

The server computing device 150 may receive electronic data and/or the like from one or more sources (e.g., the mobile computing device 125, the user computing device 200, and/or one or more databases), direct operation of one or more other devices (e.g., the mobile computing device 125 and/or the user computing device 200), contain data relating to common infectious illnesses, contain mapping data, generate plots on a map based on information generated from the common infectious illness data, contain medical provider information, contain information regarding treatment of common infectious illnesses, contain information regarding prevention against common infectious illnesses, and/or the like.

It should be understood that while the user computing device 200 is depicted as a personal computer, the mobile computing device 125 as a smartphone, and the server computing device 150 is depicted as a server, these are nonlimiting examples. More specifically, in some embodiments, any type of computing device (e.g., mobile computing device, personal computer, server, etc.) may be used for any of these components. Additionally, while each of these computing devices is illustrated in FIG. 1 as a single piece of hardware, this is also merely an example. More specifically, each of the user computing device 200, the mobile computing device 125, and the server computing device 150 may represent a plurality of computers, servers, databases, mobile devices, components, and/or the like.

In addition, while the present disclosure generally relates to computing devices, the present disclosure is not limited to such. For example, various electronic devices that may not be referred to as computing devices but are capable of providing functionality similar to the computing devices described herein, may be used. Illustrative examples of electronic devices include, for example, certain electronic medical equipment, Internet-connected electronic devices (such as certain communications devices), and/or the like may be used.

In some embodiments, the computer network 100 may further include one or more medical devices 175. Such medical devices 175 may directly obtain information from subjects, such as information related to an illness or lack thereof, and provide such information as data to be used as described herein. Illustrative examples of such medical devices 175 include, but are not limited to, blood pressure monitoring devices, thermometers, pulse oximeters, heart rate monitors, laboratory analysis equipment (e.g., equipment that receives a biological sample or the like from a subject, conducts testing, and/or determines whether the subject has a particular infection or the like from the sample) and/or the like.

It should be understood that while the embodiments depicted herein refer to a network of computing devices, the present disclosure is not solely limited to such a network. For example, in some embodiments, the various processes described herein may be completed by a single computing device, such as a non-networked computing device or a networked computing device that does not use the network to complete the various processes described herein.

In some embodiments, the network of computing devices may be a specialized network of devices that is particularly configured to provide the functionality described herein. Such a specialized network, by eliminating unnecessary components or functionality, may be able to operate more quickly and/or efficiently to determine an illness outbreak, map the illness outbreak, and notify certain individuals to take preventative action, relative to a generic computer network that allows connection between connected devices. Moreover, such functionality, despite being wholly within one or more computing devices, provides real world results that have not been observed before. More specifically, users of the devices described herein are able to be aware of common infectious illness outbreaks to react accordingly, whereas otherwise such users would not be aware of illness outbreaks and may not take the necessary precautions to prevent further spread of disease.

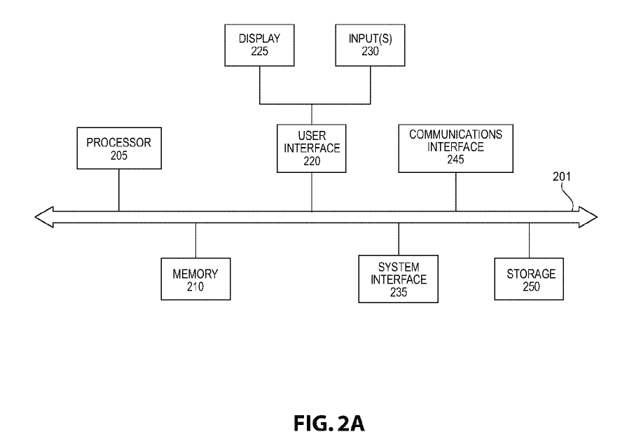

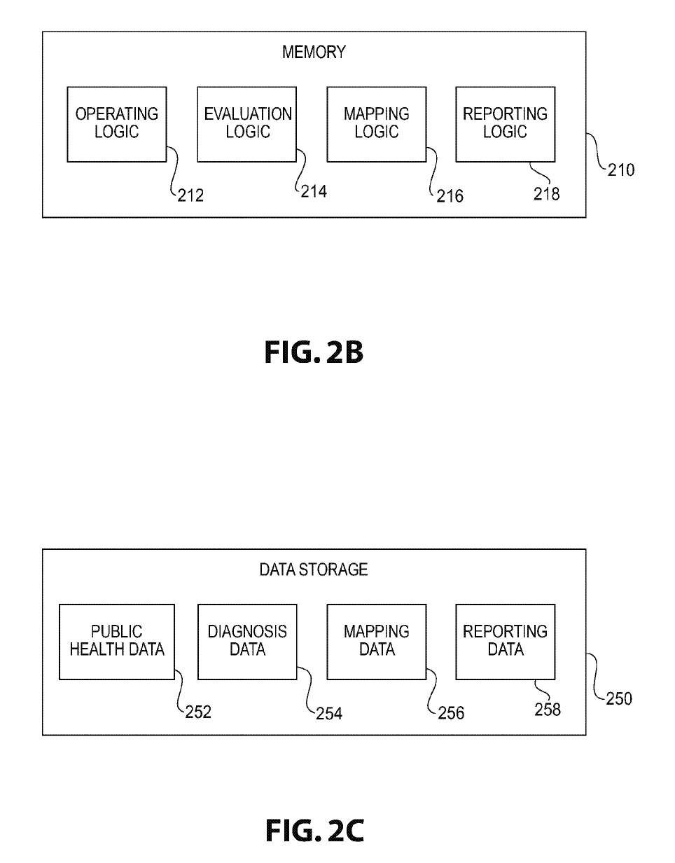

Illustrative hardware components of the user computing device 200, the mobile computing device 125, and/or the server computing device 150 are depicted in FIG. 2A. A bus 201 may interconnect the various components. A processing device 205, such as a computer processing unit (CPU), may be the central processing unit of the computing device, performing calculations and logic operations required to execute a program. The processing device 205, alone or in conjunction with one or more of the other elements disclosed in FIG. 2A, is an illustrative processing device, computing device, processor, or combination thereof, as such terms are used within this disclosure. Memory 210, such as read only memory (ROM) and random access memory (RAM), may constitute an illustrative memory device (i.e., a non-transitory processor-readable storage medium). Such memory 210 may include one or more programming instructions thereon that, when executed by the processing device 205, cause the processing device 205 to complete various processes, such as the processes described herein. Optionally, the program instructions may be stored on a tangible computer-readable medium such as a compact disc, a digital disk, flash memory, a memory card, a USB drive, an optical disc storage medium, such as a Blu-ray™ disc, and/or other non-transitory processor-readable storage media.

In some embodiments, the program instructions contained on the memory 210 may be embodied as a plurality of software modules, where each module provides programming instructions for completing one or more tasks. For example, as shown in FIG. 2B, the memory 210 may contain operating logic 212, evaluation logic 214, mapping logic 216, and/or reporting logic 218. The operating logic 212 may include an operating system and/or other software for managing components of a computing device. The evaluation logic 214 may include one or more software modules for obtaining data, generating common infectious illness information from the obtained data, and/or predicting outbreaks of common infectious illnesses. The mapping logic 216 may include one or more software modules for evaluating the common infectious illness information, plotting the information on a map, and/or predicting outbreaks of common infectious illnesses. The reporting logic 218 may contain one or more software modules for reporting outbreak information to one or more users.

Referring again to FIG. 2A, a storage device 250, which may generally be a storage medium that is separate from the memory 210, may contain one or more data repositories for storing data that is received as a result of reporting, data containing information that is received from medical devices, data that is generated as a result of determining and/or predicting a common infectious illness outbreak, data that is generated relating to mapping a common infectious illness outbreak, information regarding users that receive and/or wish to receive information regarding common infectious illness outbreaks, and/or the like. The storage device 250 may be any physical storage medium, including, but not limited to, a hard disk drive (HDD), memory, removable storage, and/or the like. While the storage device 250 is depicted as a local device, it should be understood that the storage device 250 may be a remote storage device, such as, for example, a server computing device or the like.

Illustrative data that may be contained within the storage device 250 is depicted in FIG. 2C. As shown in FIG. 2C, the storage device 250 may include, for example, public health data 252, diagnosis data 254, mapping data 256, and/or reporting data 258. Public health data 252 may include, for example, data that is obtained from or stored by public health authorities, particularly data relating to common infectious illnesses. For example, public health data 252 may include data that is stored in a database or the like maintained by local health authorities (e.g., city and/or county departments of health), state health authorities, the Centers for Disease Control (CDC), the World Health Organization (WHO), or the like. As such, the public health data 252 may be stored in a data storage device 250 that is separate from other data storage devices containing other data as described herein. Diagnosis data 254 may include, for example, data relating to one or more medical diagnoses, particularly diagnoses of common infectious illnesses. For example, diagnosis data 254 may include data that is stored in a database or the like maintained by a medical professional, a medical group, a health insurance carrier, and/or the like. In another example, diagnosis data 254 may also include data that is received directly from medical devices, such as the medical devices described herein. In some embodiments, the diagnosis data 254 may be stored in a data storage device 250 that is separate from other data storage devices containing other data as described herein. Mapping data 256 may include, for example, data generated as the result of plotting information relating to common infectious illnesses to maps for the purposes of predicting outbreaks and informing individuals, as described in greater detail herein. Reporting data 258 may include, for example, contact information, personal information, desired settings information, and/or the like from users of the systems described herein such that users that desire to receive the various information described herein are adequately provided with relevant information.

Referring again to FIG. 2A, an optional user interface 220 may permit information from the bus 201 to be displayed on a display 225 portion of the computing device in audio, visual, graphic, or alphanumeric format. Moreover, the user interface 220 may also include one or more inputs 230 that allow for transmission to and receipt of data from input devices such as a keyboard, a mouse, a joystick, a touch screen, a remote control, a pointing device, a video input device, an audio input device, a haptic feedback device, and/or the like. Such a user interface 220 may be used, for example, to allow a user to interact with the computing device or any component thereof.

A system interface 235 may generally provide the computing device with an ability to interface with one or more of the components of the computer network 100 (FIG. 1). Communication with such components may occur using various communication ports (not shown). An illustrative communication port may be attached to a communications network, such as the Internet, an intranet, a local network, a direct connection, and/or the like.

A communications interface 245 may generally provide the computing device with an ability to interface with one or more external components, such as, for example, an external computing device, a remote server, and/or the like. Communication with external devices may occur using various communication ports (not shown). An illustrative communication port may be attached to a communications network, such as the Internet, an intranet, a local network, a direct connection, and/or the like.

It should be understood that the components illustrated in FIGS. 2A-2C are merely illustrative and are not intended to limit the scope of this disclosure. More specifically, while the components in FIGS. 2A-2C are illustrated as residing within the server computing device 150, the mobile computing device 125, or the user computing device 200, these are nonlimiting examples. In some embodiments, one or more of the components may reside external to the server computing device 150, the mobile computing device 125, and/or the user computing device 200. Similarly, one or more of the components may be embodied in other computing devices not specifically described herein.

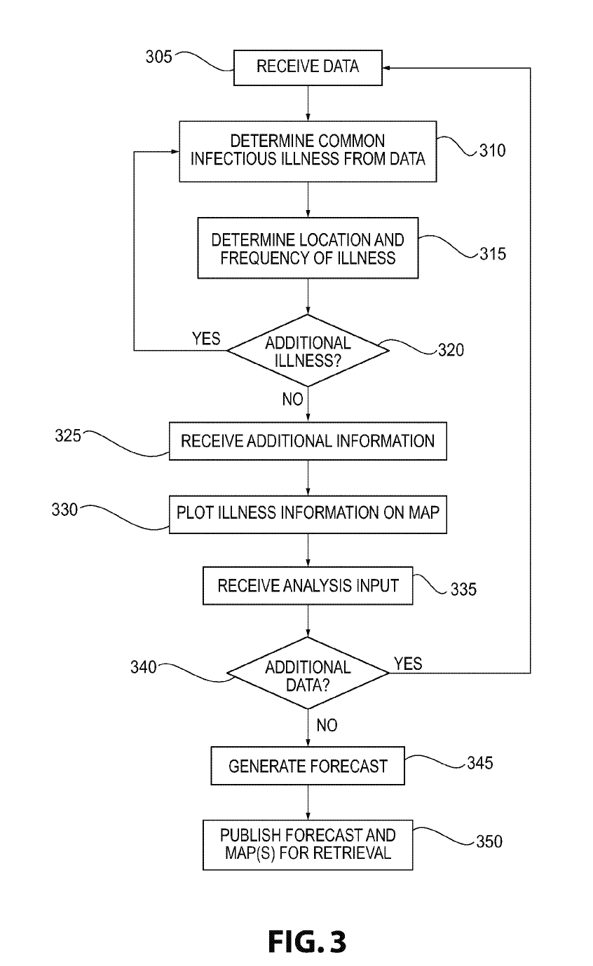

Referring now to FIG. 3, a method of tracking and predicting common infectious illnesses is described. Such a method may be completed by one or more devices and/or systems, such as, for example, the devices and/or systems described herein.

At step 305, data may be received. The data may be received from any database that includes health related data, particularly data relating to common infectious illnesses. In some embodiments, the data may be received from a cloud based health data provider, a data source, a data analyst, and/or the like.

In some embodiments, such databases may include databases that are maintained by medical personnel (e.g., hospital network and/or doctor's office databases) and/or medical insurance carrier databases. However, the present disclosure is not limited to such, and the data may be received from other databases. The data may generally be received by accessing the databases and obtaining the data therefrom. In some embodiments, data may be received from various medical devices, such as, for example, the medical devices 175 described herein with respect to FIG. 1. The data may be received directly from the various medical devices or may be passed through the one or more databases before being received.

In some embodiments, the data may be received continuously. In other embodiments, the data may be received at various intervals. For example, the data may be received as a compilation of information that is provided, for example, on a daily basis, a weekly basis, a biweekly basis, a monthly basis, and/or the like. In some embodiments, data may be automatically pushed such that it is received as described with respect to step 305. In other embodiments, the data may be received in response to a request to obtain the data. That is, a computing device (such as, for example, the server computing device 150 depicted in FIG. 1) may transmit a request to an external source (e.g., a remote database, the medical device 175 depicted in FIG. 1, and/or the like), where the request includes a request for particular data held by the source, and the source provides the particular data in response to the request.

The data that is received according to step 305 generally relates to common infectious illness diagnoses. That is, the data may include information regarding a common infectious illness diagnosis, the type of illness, the severity of illness, the onset of the illness, the date of diagnosis, the treatment provided, medications prescribed, and/or the like. In some embodiments, the data may contain the actual diagnosis made by medical personnel. In other embodiments, the data may not provide the actual diagnosis, but may be data that was used by medical personnel to make the diagnosis. The data may be provided in the aggregate and may not contain any patient identifying information, so as to protect patients' privacy. That is, the data may contain information about each diagnosis that was made, how it was made (i.e., data relating to testing that was completed, etc.), and/or the like, but may not contain any personally identifying information, such as a subject's name, birthdate, social security number, address, and/or the like. In addition, the data may not contain information that could potentially be used to identify a particular individual (i.e., specific demographic information about the subject, together with the subject's zip code or the like that could potentially be used to identify the subject). An illustrative example of the data includes ICD-10 code data, such as ICD-10 code data that is transmitted from medical personnel to health insurance providers, medical billing companies, public health organizations, and/or the like. ICD-10 generally refers to the 10th revision of the International Statistical Classification of Diseases and Related Health Problems (ICD), which is a medical classification list provided by the World Health Organization (WHO). The ICD-10 contains codes for diseases, signs, symptoms, abnormal findings, complaints, social circumstances, and external causes of injury or diseases. ICD-10, as used herein, includes various sub-classifications and/or various national modifications, such as, for example, the U.S. ICD-10 Clinical Modification (ICD-10-CM), and the U.S. ICD-10 Procedure Coding System (ICD-10-PCS). Other details of the ICD-10 codes, as well as modifications thereof, should generally be understood. Use of ICD-10 code data for the purposes of predicting common infectious illness outbreaks as described herein may be advantageous over use of other types of medical coding data, such as ICD-9 data, because it is more robust and more accurate for the purposes of determining outbreaks. It should be understood that ICD-10 code data is merely one illustrative example, and other data, including data now known or later developed, may also be used without departing from the scope of the present disclosure.

At step 310, the common infectious illness may be determined from the data. Determining the common infectious illness may include analyzing the data and extracting a diagnosis from the data (e.g., a diagnosis made by medical personnel and provided with the data). For example, the data may contain ICD-10 code J00, which is the code for acute nasopharyngitis, which is also referred to as the common cold. As such, determining at step 310 may include analyzing the data to discover code J00 and using a lookup table or the like (e.g., accessing a supplemental database) to extract/determine the corresponding diagnosis (acute nasopharyngitis). If ICD-10 codes for other diagnoses that are not related to common infectious illnesses (e.g., code F03, which is the code for unspecified dementia) are discovered, such codes may be ignored. In such instances, the data may be further analyzed for other codes related specifically to common infectious illnesses.

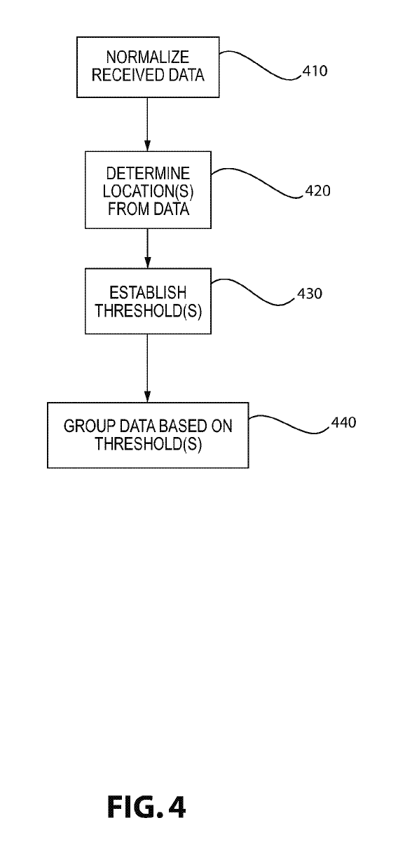

Once the common infectious illness has been determined, the location and frequency of the illness may be determined at step 315. Such a determination may generally include analyzing additional information contained within the data that relates to location (e.g., location of medical personnel where the diagnosis was made), determining from the data the number of times the illness has been diagnosed, determining the location of the medical facility at which the illness was diagnosed, determining the location (e.g., zip code) of the subject that was diagnosed (if available), and/or the like. FIG. 4 provides additional detail regarding the determination of location and frequency. For example, at step 410, the data that was received may be normalized.

Normalizing the data may include projecting to correct for delays in receiving the data. That is, as described herein, data may be received periodically, which may result in data that encompasses a particular time period (e.g., data encompassing 3 days' worth of diagnoses), and receipt may be delayed (e.g., data may be received 7-9 days after it is generated). As such, it may be necessary to project total cases for a given week based on the received data, and update the determination once the data corresponding to the remainder of the week is received.

In some embodiments, normalizing the data may include adjusting the number of cases to cases per 100,000 people such that the cases can be compared nationally. For example, if 10 cases of the common cold are reported in a given week for a population of 1,000 individuals, this may be adjusted to correspond to the number of cases that likely would be present in a population of 100,000 individuals (i.e., 10,000 cases). In addition, the number of cases may be adjusted based on particular age ranges of subjects (e.g., 0-1 years old, 2-4 years old, 5-12 years old, 13-17 years old, 18-22 years old, 23-54 years old, 55+ years old). Such information may be based on data received from other databases, such as, for example, U.S. census data. While a population of 100,000 individuals is used herein, it should be understood that such a number is merely illustrative, and normalizing may include adjusting the number of cases as appropriate without departing from the scope of the present disclosure.

In some embodiments, the data may be normalized to account for incubation periods of common infectious illnesses such that, when the data is reported as described in greater detail herein, it reflects current illness levels rather than historical illness levels. It should be understood that particular infectious illnesses may have an incubation period in which a subject has the disease, but is not exhibiting any symptoms. For example, the common cold may have an incubation period of about 24-72 hours. In another example, mononucleosis may have an incubation period of about 4-6 weeks. As such, data smoothing may be used to account for these incubation periods to ensure that the diagnosis information corresponds to when an individual is actually infected. For example, current risks may be calculated from data received from more than the previous week, such as from the previous two weeks, the previous three weeks, the previous 4 weeks, and/or the like.

Similar to the incubation period, in some embodiments, the data may be normalized to account for periods wherein an individual is infectious (i.e., contagious) with a common infectious illness such that, when the data is reported as described in greater detail herein, it accurately reflects current illness levels. It should be understood that an infectious individual may be contagious (i.e., able to spread the disease to others), but may not necessarily be exhibiting any symptoms. As such, data smoothing may be used to account for these infectious periods to ensure that the diagnosis information corresponds to when an individual is actually infected.

At step 420, the various locations of the common infectious illnesses may be determined. Such a determination may include projecting a patient location based on the location of the medical facility (e.g., a doctor's office or the like). That is, as described herein, the data that is received may include location data corresponding to the medical facility where the diagnosis was made. In some embodiments, the received data may specify a general area of the location, which may be based on, for example, a postal code or the like. For example, in the United States, the data may specify a ZIP code, such as a 9 digit ZIP code, a 5 digit ZIP code, or may provide the first 3 digits of a 9 or 5 digit ZIP code. Since the first three digits in a 5 or 9 digit ZIP code in the United States may refer to a relatively large geographical area (e.g., a large metropolitan area, a region of a particular state, or the like), and subjects may travel out of their home ZIP code to see medical personnel, it may be necessary to make a series of assumptions to ensure the location data is correctly determined. Such assumptions may be based on doctor per population numbers in particular ZIP codes. For example, if a median number of medical service providers in a particular zip code is 50 out of 100 and a particular ZIP code has about 60 or greater, such a ZIP code may be assumed to receive subjects from an area outside the ZIP code. In contrast, if a particular ZIP code has about 40 or less, such a ZIP code may be assumed to send subjects to an area outside the ZIP code. If the above two ZIP codes are adjacent to one another, they may each be adjusted to be closer to the median. As such, particular cases may be moved to ZIP codes of surrounding areas based on a typical distance traveled by subjects to see medical personnel. For example, if a typical distance that a subject will travel to visit medical personnel is about a 20 mile radius from the subject's home, then the cases may be moved to ZIP codes of surrounding areas that are within 20 miles of where the case was reported. Therefore, the cases per ZIP code may be normalized in accordance with a particular medical personnel density. Such distribution may also be based on obtained data relating to population density (i.e., subjects may travel less in more population dense areas than subjects that are in less population dense areas.

In some embodiments, to ensure that mapping (as described in greater detail herein) accurately reflects the received data, it may be necessary to implement one or more mapping classification techniques to establish one or more thresholds at step 430. Such a mapping classification technique may generally be used to compare current data with historical data to determine severity of the common infectious illness, as described in greater detail herein. In addition, such a mapping classification technique may be completed for each established area (e.g., each area containing a particular ZIP code, a grouping of ZIP codes, a quantile, or the like). One example of a mapping classification technique may be a Jenks natural break classification technique. The Jenks natural breaks classification technique, which may also be referred to as the Jenks optimization method, is a data clustering method designed to determine the best arrangement of values into different classes. This may be completed by seeking to minimize each class's average deviation from a class mean, while maximizing each class's deviation from the means of the other groups. That is, the technique seeks to reduce the variance within classes and maximize the variance between classes. The Jenks natural breaks classification technique is only one illustrative technique. Other classification techniques should generally be understood and are included within the scope of the present disclosure. As a result of applying the classification technique, the data may be grouped based on the one or more established thresholds at step 440.

Referring again to FIG. 3, the various determinations as described above with respect to steps 310 and 315 may be completed for each common infectious illness that is obtained from the received data. As such, a determination may be made at step 320 as to whether additional common infectious illnesses are present in the data. If so, the process may return to step 310 and may repeat steps 310-320 as many times as needed to ensure all common infectious illnesses are accounted for. Once all of the common infectious illnesses have been determined and a location/frequency have been determined, the process may proceed to step 325.

At step 325, additional information may be received, such as supplemental information that may be useful in predicting an outbreak. Such additional information is not limited by this disclosure. A nonlimiting example of additional information may include information obtained from public health sources. The additional information may allow for a more accurate plotting of the information on a map, as described herein.

Once all of the information has been determined, the illness information may be plotted on a map at step 330. The plots may be based on the various determinations described herein with respect to steps 310 and 315, as well as steps 410-440 (FIG. 4). Plotting the information on a map may allow a user viewing the map to determine locations where the illness is occurring, as well as an intensity of the illness (e.g., a particular area that contains 10 cases of the same illness has a higher intensity than a particular area that contains 1 case of an illness).

At step 335, an analysis input may be received. Such an analysis input may generally include a predictive analysis of a common infectious illness outbreak based on the data that was received, the information that was obtained therefrom, and the information plotted on the map. The analysis may be a result of a computer process that is specifically configured to provide a prediction of an outbreak of a common infectious illness, or may be an input that is received from an individual, such as an epidemiology expert, a medical professional, and/or the like. In embodiments where a computer process is used, any predictive analytics algorithm may be implemented. It should generally be understood that predictive analytics is an area of statistics that deals with extracting information from data and using it to predict trends and behavior patterns. The core of predictive analytics relies on capturing relationships between explanatory variables and the predicted variables from past occurrences, and exploiting them to predict the unknown outcome. As such, the type of predictive analytics algorithm that is used is not limited by this disclosure.

In some embodiments, as described herein, an accurate prediction, forecasting, and reporting of common infectious illness outbreaks may be based on historic data such that trends can be determined and analyzed. As such, the systems and methods described herein may be particularly configured to periodically obtain data over a period of time. For example, data may be obtained on a daily basis, a weekly basis, a monthly basis, or the like. As such, a determination is made at step 340 as to whether additional data is needed to accurately generate a forecast of a common infectious illness outbreak. If additional data is needed (e.g., because the data was last collected before a period of time has elapsed), the process may return to step 305 such that additional data is received.

If sufficient data has been collected to generate a forecast, the forecast may be generated at step 345. Generating the forecast may include comparing the forecast to a moving average. For example, forecasts may be seasonal forecasts, weekly forecasts, and/or the like. A seasonal forecast may be completed, for example, by generating an 8 week moving average for a particular area, and then comparing the moving average to the current week. If the current week is greater than the 8 week moving average, it may be indicative of an increasing severity period. For shorter term forecasts (e.g., a 1 week forecast), severity increases of a particular percentage may be evaluated and compared to a past time period, such as, for example, the previous week, the same time period in the previous year, and/or the like.

The generated forecast may be published (i.e., reported) at step 350. As such, a user viewing the generated and published/reported forecast should be able to see what type of common infectious illness outbreak is occurring in a particular area, is predicted to occur, the intensity of the outbreak, whether the outbreak is moving in or out of an area, and/or the like. The information may be provided to the users via any user interface, such as the user interface described herein. As such, a user may access a website, a mobile app, or the like to obtain information regarding the prediction and/or the forecast.

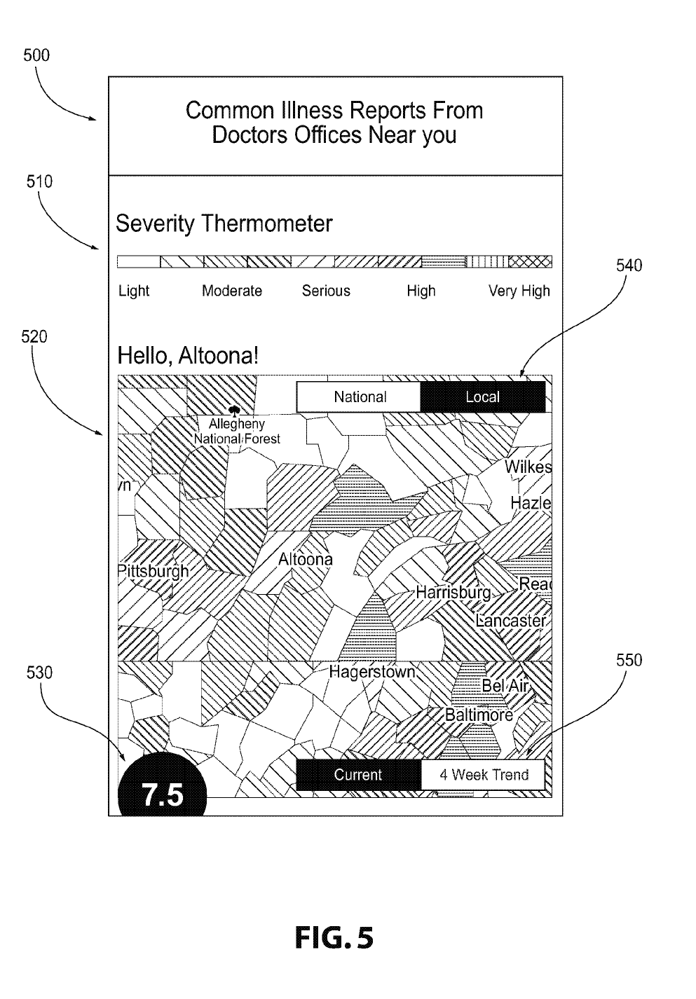

For example, as shown in FIG. 5, an illustrative map user interface 500 may include a map 520 that is shaded, colored, or the like to correspond to a severity of a particular common infectious illness, as indicated by a severity thermometer legend 510. The map user interface 500 may allow a user to zoom in/out on the map to show national or local details at selection box 540, pan the map to move to a different area, select current severity or previous trend at selection box 550. While selection box 550 depicts current and 4 week trends, this is merely illustrative. Other time periods for trends may be used without departing from the scope of the present disclosure, such as, for example, a 1 week trend, a 2 week trend, a 3 week trend, a 5 week trend, a 6 week trend, a 7 week trend, an 8 week trend, or the like. In addition, if a user selects a particular area on the map 520, it may provide a severity level 530 of the selected area. The severity level 530 may be a numerical indicator that provides the user with the frequency of cases. For example, the severity level may rank the frequency of cases on a scale of 1 to 10, where 10 is the most severe frequency (i.e., the most amount of cases).

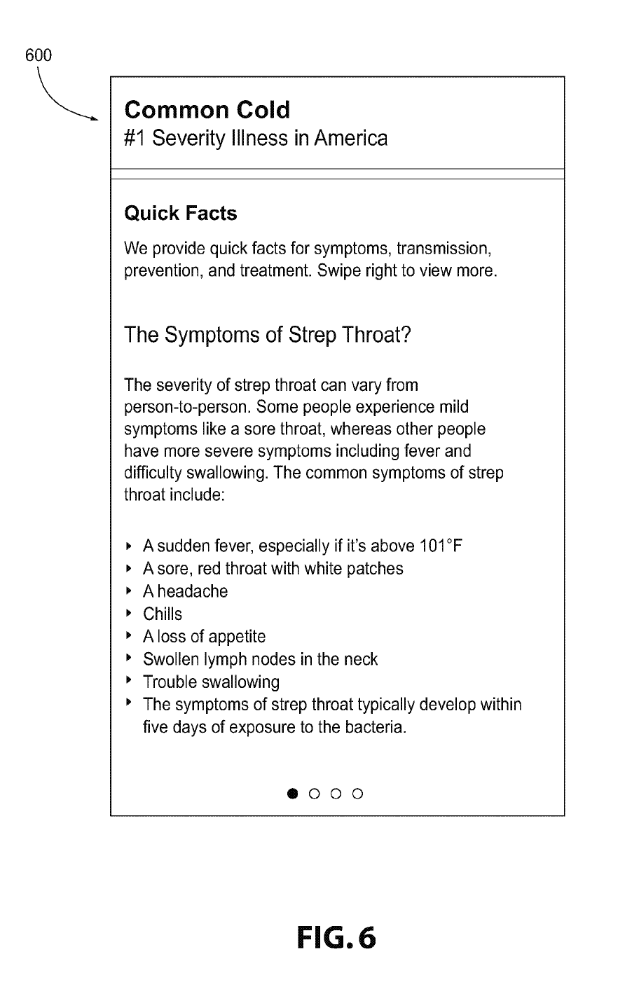

Other information that may be provided to a user may include a description user interface 600, as shown in FIG. 6. Such a description user interface 600 may provide general information about a particular common infectious illness, including how common it is relative to other illnesses, other common infectious illnesses, and/or the like, various quick facts about the illness, various symptoms of the illness, and/or the like.

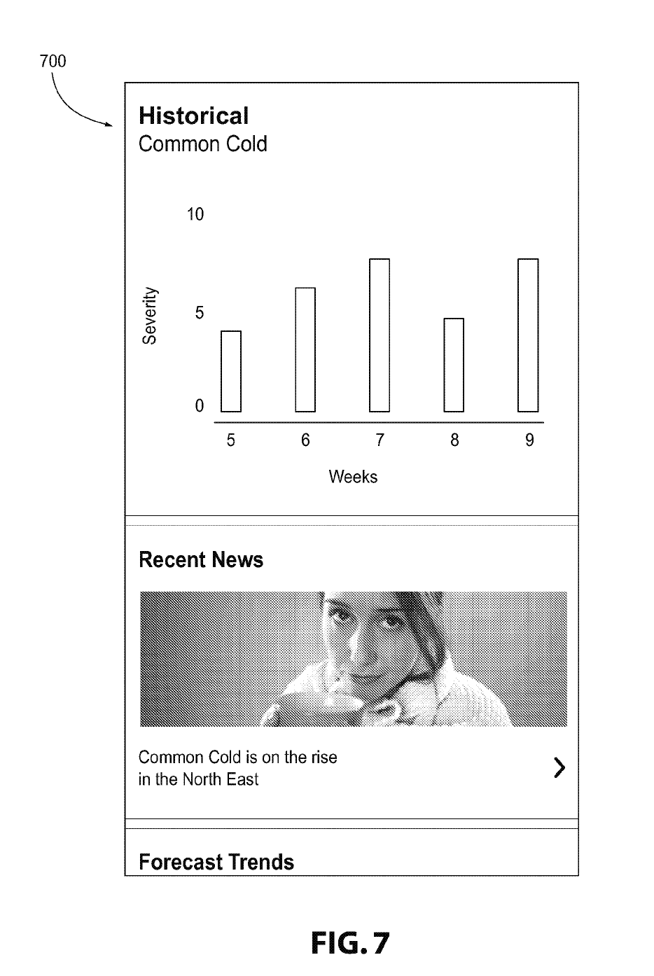

In addition, a user may also be provided with a historical trends user interface 700 as shown in FIG. 7. The historical trends may show information such as, for example, how severe a particular common infectious illness was over the course of past weeks. Such information may potentially be useful to a user in determining whether an illness is on the rise (i.e., becoming more severe), when an illness is decreasing (i.e., becoming less severe), when an illness severity is remaining flat, and/or the like. Severity may generally be based on historical trends, such as, for example, based on a previous period of time (e.g., a previous week, previous two weeks, previous season), a comparison to the same time period in a previous year, and/or the like. While the historical trends user interface 700 depicted in FIG. 7 is a bar chart, this is merely illustrative. Other charts that may convey the same or similar information to a user may also be used without departing from the scope of the present disclosure. In addition, the historical trends user interface 700 may allow a user to specify a particular area for which to observe a change in trend. For example, the user may select a region having a radius of about 7.5 miles, a radius of about 15 miles, or the like. In some embodiments, the user may select particular regions, particular groups of regions, particular countries, and/or the like.

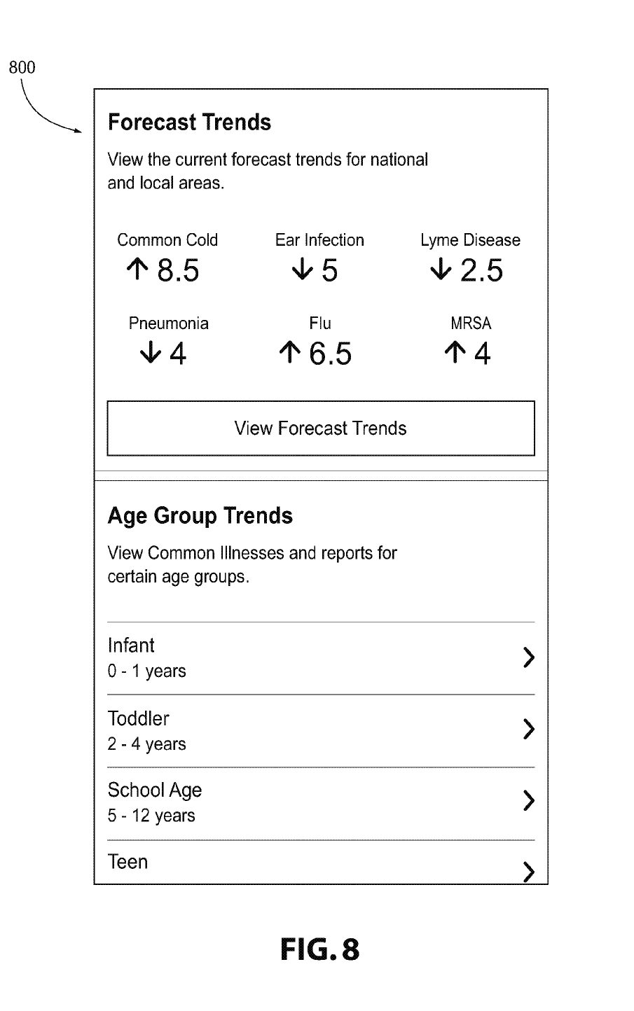

As mentioned above, historical trends may also be presented in other manners. For example, a forecast trends user interface 800 may display a current forecast for various common infectious illnesses, the current severity level of the illness for a given area (as indicated by the numbers in FIG. 8), whether the illness severity is on the rise or decreasing (as indicated by the upwards and downwards pointing arrows), and/or the like. While the common cold, ear infections, Lyme disease, pneumonia, influenza, and methicillin resistant Staphylococcus aureus (MRSA) infections are shown in FIG. 8, these are merely illustrative. As such, other common infectious diseases may also be displayed without departing from the scope of the present disclosure. In some embodiments, the forecast trends user interface 800 may be user adjustable such that a user can specify which common infectious illnesses he/she wishes to view.

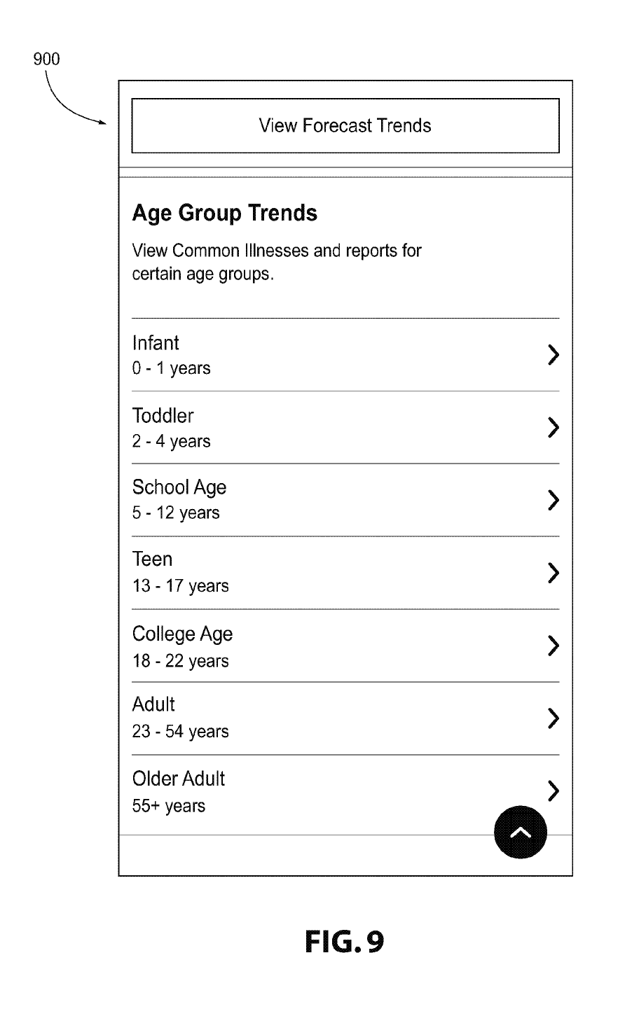

The bottom of FIG. 8 and FIG. 9 depict an age group trend user interface 900 that can be used by a user to determine various trends for particular age groups. While infants (0-1 years old), toddlers (2-4 years old), school age children (5-12 years old), teens (13-17 years old), college age adults (18-22 years old), adults (23-54 years old), and older adults (55+ years old) are depicted, these are merely illustrative. Other age ranges or categorizations based on age may also be used without departing from the scope of the present disclosure.

It should be understood that the various user interfaces depicted in FIGS. 5-9 are merely illustrative, and other user interfaces that depict data in a different manner are also included within the scope of the present disclosure.

It should now be understood that the embodiments described herein are generally directed to systems and methods that obtain data from various health related sources, determine common infectious illness information from the data, determine a location and/or a frequency of the common infectious illnesses, plot the common infectious illness information on a map, and predict an outbreak of the common infectious illness based on the plots on the map. The data may be collected over a period of time such that movement in the plots can be observed (e.g., certain areas are seeing an increase in a particular illness over a period of time). As a result, users viewing the collected data as plotted in a chart, a map, or the like, can determine a potential for contracting a common infectious illness and take necessary steps to prevent contraction of the illness.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue. While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A computer-based method of tracking a common infectious illness and disseminating information regarding the common infectious illness to a plurality of users via one or more of a mobile device and a user computing device, the method comprising:
receiving, by a processing device, data from one or more electronic sources;
determining, by the processing device, the common infectious illness from the data;
determining, by the processing device, one or more of a location and a frequency of the common infectious illness from the data;
plotting, by the processing device, information relating to the common infectious illness on a map, wherein the information comprises a current severity of the common infectious illness in a particular area and predicted trend of the severity of the common infectious illness; and
providing, by the processing device, the map to the plurality of users.

2.-5. (canceled)

6. The computer-based method of claim 1, wherein the data comprises one or more of a medical personnel diagnosis, a type of illness, a severity of the illness, an onset of the illness, a date of the medical personnel diagnosis, a provided treatment, and a prescribed medication.

7.-8. (canceled)

9. The computer-based method of claim 1, wherein determining the one or more of the location and the frequency of the common infectious illness from the data comprises analyzing additional information contained within the data that relates to a medical personnel location, the medical personnel location being a location where a diagnosis of the common infectious illness was made.

10. The computer-based method of claim 1, wherein determining the one or more of the location and the frequency of the common infectious illness from the data comprises analyzing additional information contained within the data to determine a number of cases relating to the common infectious illness in a particular location.

11. The computer-based method of claim 1, wherein determining the one or more of the location and the frequency of the common infectious illness from the data comprises normalizing the data by projecting to correct for delays in receiving the data.

12. The computer-based method of claim 1, wherein determining the one or more of the location and the frequency of the common infectious illness from the data comprises normalizing the data by adjusting the number of cases for the common infectious illness to cases per 100,000.

13. The computer-based method of claim 1, wherein determining the one or more of the location and the frequency of the common infectious illness from the data comprises normalizing the data to account for an incubation period of the common infectious illness.

14. The computer-based method of claim 1, further comprising implementing, by the processing device, one or more mapping classification techniques on the data prior to plotting the information on the map.

15. (canceled)

16. The computer-based method of claim 1, further comprising receiving, by the processing device, a predictive analysis of a common infectious illness outbreak based on the data, the information, and the map.

17. The computer-based method of claim 1, wherein the current severity of the common infectious illness comprises a numerical indicator that is based on the number of cases of the common infectious illness in a particular area.

18. The computer-based method of claim 1, wherein the predicted trend of the severity of the common infectious illness comprises an indicator of whether the severity of the common infectious illness is on the rise, whether the severity of the common infectious illness is decreasing, or whether the severity of the common infectious illness is remaining stable.

19. A system for tracking a common infectious illness and disseminating information regarding the common infectious illness to a plurality of users via one or more of a mobile device and a user computing device, the system comprising:
a processing device; and
a non-transitory, processor-readable storage medium, the non-transitory, processor readable storage medium comprising one or more programming instructions thereon that, when executed, cause the processing device to:
receive data from one or more electronic sources;
determine the common infectious illness from the data;
determine one or more of a location and a frequency of the common infectious illness from the data;
plot information relating to the common infectious illness on a map, wherein the information comprises a current severity of the common infectious illness in a particular area and predicted trend of the severity of the common infectious illness; and
provide the map to the plurality of users.

20.-26. (canceled)

27. The system of claim 19, wherein the one or more programming instructions that, when executed, cause the processing device to determine the one or more of the location and the frequency of the common infectious illness from the data further cause the processing device to analyze additional information contained within the data that relates to a medical personnel location, the medical personnel location being a location where a diagnosis of the common infectious illness was made.

28. The system of claim 19, wherein the one or more programming instructions that, when executed, cause the processing device to determine the one or more of the location and the frequency of the common infectious illness from the data further cause the processing device to analyze additional information contained within the data to determine a number of cases relating to the common infectious illness in a particular location.

29. The system of claim 19, wherein the one or more programming instructions that, when executed, cause the processing device to determine the one or more of the location and the frequency of the common infectious illness from the data further cause the processing device to normalize the data by projecting to correct for delays in receiving the data.

30. The system of claim 19, wherein the one or more programming instructions that, when executed, cause the processing device to determine the one or more of the location and the frequency of the common infectious illness from the data further cause the processing device to normalize the data by adjusting the number of cases for the common infectious illness to cases per 100,000.

31. The system of claim 19, wherein the one or more programming instructions that, when executed, cause the processing device to determine the one or more of the location and the frequency of the common infectious illness from the data further cause the processing device to normalize the data to account for an incubation period of the common infectious illness.

32.-33. (canceled)

34. The system of claim 19, further comprising one or more programming instructions that, when executed, cause the processing device to receive a predictive analysis of a common infectious illness outbreak based on the data, the information, and the map.

35. (canceled)

36. The system of claim 19, wherein the predicted trend of the severity of the common infectious illness comprises an indicator of whether the severity of the common infectious illness is on the rise, whether the severity of the common infectious illness is decreasing, or whether the severity of the common infectious illness is remaining stable.

37. A computer-based method of tracking a plurality of common infectious illnesses and disseminating information regarding each common infectious illness from the plurality of common infectious illnesses to a plurality of users via one or more of a mobile device and a user computing device, the method comprising:
receiving, by a processing device, data from one or more electronic sources;
determining, by the processing device, each common infectious illness from the data;
determining, by the processing device, one or more of a location and a frequency of each common infectious illness from the data;
plotting, by the processing device, information relating to each common infectious illness on a map, wherein the information comprises a current severity of each common infectious illness in a particular area and predicted trend of the severity of each common infectious illness; and
providing, by the processing device, the map to the plurality of users.
